(19) United States
(12) Plant Patent Application Publication
(10) Pub. No.: US 2003/0097696 P1
(43) Pub. Date: May 22, 2003

(54) PELARGONIUM PLANT NAMED 'SARAH DON'

(76) Inventor: Roger Jones, Acresford (GB)

Correspondence Address: PLANT HAVEN, INC., 121 W. PUEBLO STREET, SUITE 14, P.O. BOX 3056, SANTA BARBARA, CA, 93130-3056, US

(21) Appl. No.: 10/002,426

(22) Filed: Nov. 21, 2001

(51) Int. Cl. A01H 5/00

(52) U.S. Cl. PLT/325

(57) ABSTRACT

A new cultivar of Pelargonium plant named ‘Sarah Don’ that is characterized by golden-yellow and lime-green variegated foliage and mauve flowers with dark purple-pink markings. In combination these traits set ‘Sarah Don’ apart from all other existing varieties of Pelargonium known to the inventor.

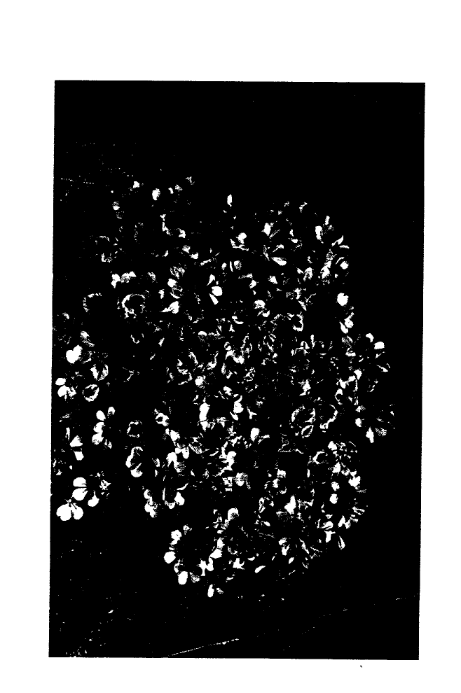

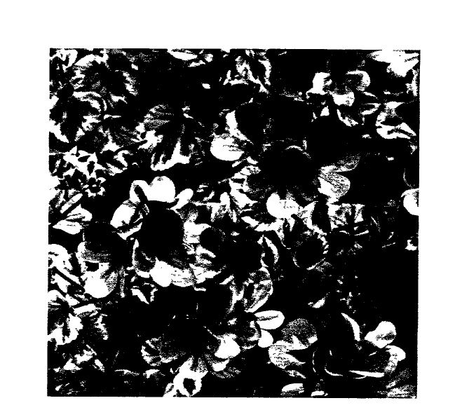

### BACKGROUND OF THE INVENTION

The present invention relates to a new and distinct cultivar of geranium that is grown as an ornamental for its yellow and green variegated foliage. The new cultivar is known botanically as Pelargonium×domesticum and will be referred to hereinafter by the cultivar name ‘Sarah Don’.

‘Sarah Don’ was discovered by the inventor as a sport branch at the base of a commercially grown Pelargonium ‘Darmsden’ (unpatented) in a cultivated area of Hampshire, England. On Nov. 12, 1998 the inventor selected ‘Sarah Don’ for its unique golden-yellow and lime-green variegated foliage. The parent plant, Pelargonium ‘Darmsden’ does not exhibit any variegation.

‘Sarah Don’ is from a group of Pelargonium commonly referred to as Angel Pelargonium. Within this group the flowers are smaller and more numerous than in any other group of Pelargonium.

‘Sarah Don’ was first asexually propagated by the inventor in 1998 in a cultivated area of Hampshire, England. The method used for asexual propagation was vegetative cuttings. Since that time the characteristics of the new cultivar have been determined stable and are reproduced true to type in successive generations.

### SUMMARY OF THE INEVNTION

The following traits have been repeatedly observed and represent the distinguishing characteristics of the new cultivar ‘Sarah Don’. In combination these traits set ‘Sarah Don’ apart from all other Pelargonium known to the inventor. ‘Sarah Don’ has not been tested under all possible conditions and phenotypic differences may be observed with variations in environmental, climatic and cultural conditions, however, without any variance in genotype.

1. Pelargonium ‘Sarah Don’ exhibits golden-yellow and lime-green variegated foliage.

2. Pelargonium ‘Sarah Don’ flowers profusely and exhibits mauve flowers with dark purple-pink markings.

3. Pelargonium ‘Sarah Don’ grows to 40 cm. in height and 72 cm. in width at maturity.

4. Pelargonium ‘Sarah Don’ exhibits a dense, mounding habit.

5. Pelargonium ‘Sarah Don’ is hardy to USDA Zone 8.

### BRIEF DESCRIPTION OF THE DRAWINGS

The accompanying drawings illustrate the distinguishing traits of the new cultivar ‘Sarah Don’. The plants in the photographs were grown in Arroyo Grande, Calif. in one-gallon containers. The plants were 12 months old at the time and were photographed in July. Sheet 1 illustrates an entire plant from a side perspective. Sheet 2 shows a closeup view of the flowers and the foliage. All photographs were made using conventional techniques and although colors may appear different from actual colors due to light reflectance they are as accurate as possible by conventional photography.

### BOTANICAL DESCRIPTION OF THE PLANT

The following is a detailed description of the new Pelargonium named ‘Sarah Don’. Data was collected in Arroyo Grande Calif. from plants grown in one-gallon containers. The color determinations are in accordance with the RHS Color Chart of The Royal Horticultural Society, London, England, except where general color terms of ordinary dictionary significance are used. The growing requirements are similar to other Pelargonium.

Botanical classification: Pelargonium×domesticum ‘Sarah Don’.

Species: domesticum.

Commercial classification: Perennial.

Common name: Geranium.

Use: Ornamental for containers and as a bedding plant.

Cultural requirements: Plant in full sun or partial shade and fast-draining soil.

Parentage: Pelargonium ‘Sarah Don’ is a sport of Pelargonium ‘Darmsden’. The parent plant is Pelargonium ‘Darmsden’.

Plant description:

Bloom period.—Spring and summer, extending into fall.

Plant habit.—Erect, dense and mounding.

Height.—Reaches 40-50 cm. in height at maturity.

Width.—Reaches 40-60 cm. in width at maturity.

Hardiness.—USDA Zone 8.

Propagation.—Propagation is accomplished using vegetative cuttings.

Time to develop roots.—10-14 days, when rooted on a heated bench.

Crop time.—Eight weeks are needed to produce a 4-inch container from a rooted cutting and two additional months are needed to produce a finished one-gallon container.

Disease problems.—Using contaminated compost can lead to blackleg and Pelargonium collapse.

Stem:

Shape.—Cylindrical.

Stem color.—144B.

Stem dimensions.—35 cm. in height and 0.75 cm. in diameter.

Stem surface.—Pubescent with whitish hairs up to 3 mm in length.

Basal stem shape.—Columnar.

Basal stem color.—146A.

Branching.—Basal branching. Branching from all nodes once flowering is initiated.

Internode length.—1.25-3 cm. between nodes.

Branching habit.—Basal branching with erect stems.

Foliage:

Type.—Evergreen.

Shape.—Reniform to trilobed leaves.

Division.—Simple.

Apex.—Acute.

Base.—Cordate.

Venation.—Palmate with veins depressed on adaxial surface and protruding on abaxial surface.

Vein color (adaxial surfaces).—147A.

Vein color (abaxial surfaces).—147C.

Margins.—Undulate and biserrate.

Texture.—Flexible.

Arrangement.—Alternate and spiral.

Attachment.—Petiolate.

Surfaces (adaxial and abaxial).—Rugose and pubescent.

Mature leaf dimensions.—2-3 cm. in length and 3-4.25 mm. in width.

Young leaf dimensions.—1.25 cm. in length and 1.75 cm. in width.

Leaf color (adaxial surface).—Center is 143A,B and margin is 11B,C.

Leaf color (abaxial surface).—Center is 147C and margin is 11A.

Petiole shape.—Sulcate.

Petiole color.—144A.

Petiole surface.—Soft whitish hairs up to 3 mm in length.

Petiole dimensions.—2 cm. in length and 1 mm. in diameter. Petiole can extend up to 12 cm under low light conditions.

Flowers:

Inflorescence.—Corymb.

Dimensions of inflorescence.—4 cm. in width and 4 cm. in height.

Shape.—Funnelform and apopetalous.

Flower dimensions.—20-27 mm. in width and 10-12 mm. in height.

Persistent or self-cleaning.—Self-cleaning.

Aspect.—Facing upward.

Sexuality.—Bisexual.

Bud dimensions.—0.25 cm. in width and 0.30 cm. in length.

Bud shape.—Oval.

Bud color.—144A.

Flower color.—Pale color of lower petals is 76C, dark upper petals are 74A, darkest spots on petals are 79A, and dark veining on petals is 72A.

Petals.—Five in number (2 upper and 3 lower).

Fused or unfused.—Unfused.

Petal dimensions.—Lower pale colored petals are 2 cm. in length and 1 cm. in width, and upper dark colored petals are 2 cm. in length and 1.50 cm. in width.

Petal margin.—Entire.

Petal surface.—Glabrous.

Calyx dimensions.—1 cm. in height and 1 cm. in diameter.

Calyx surface.—Pubescent.

Sepals.—Five in number.

Sepals fused or unfused.—Unfused.

Sepal margin.—Entire.

Sepal color.—138B.

Sepal surface.—Pubescent.

Sepal apex.—Aristiculate.

Peduncle dimensions.—1 cm. in length and 2 mm. in diameter.

Peduncle color.—184A.

Peduncle surface.—Pubescent.

Reproductive organs:

Stamens.—Seven in number.

Stamen color.—74D.

Stamen dimensions.—1.50 cm. in length and 0.50 mm. in diameter.

Pistil.—One.

Pistil dimensions.—9 mm. in length and 0.50 mm. in width.

Pistil color.—72A.

Ovary position.—Superior.

Ovary color.—138D.

Ovary shape.—Oval.

Ovary dimensions.—3 mm. in height and 2 mm. in diameter.

Seed production: Seed production is either absent or it is so sporadic that it has not been observed.

1. A new and distinct cultivar of Pelargonium plant named ‘Sarah Don’ as described and illustrated herein.
